(19) United States
(12) Patent Application Publication
(10) Pub. No.: US 2009/0131303 A1
(43) Pub. Date: May 21, 2009

(54) METHODS AND COMPOSITIONS FOR TREATING DRY EYE

(76) Inventors: BOR-SHYUE HONG, Arlington, TX (US); DAVID L. MEADOWS, Colleyville, TX (US)

Correspondence Address: ALCON, IP LEGAL, TB4-8, 6201 SOUTH FREEWAY, FORT WORTH, TX, 76134, US

(21) Appl. No.: 12/270,406

(22) Filed: Nov. 13, 2008

Related U.S. Application Data
(60) Provisional application No. 60/988,623, filed on Nov. 16, 2007.

(51) Int. Cl. A61K 38/16; A61K 31/736; A61K 38/39; A61P 27/16; A61K 38/00

(52) U.S. Cl. 514/6; 514/54; 514/12

(57) ABSTRACT

The present invention is directed to ophthalmic compositions containing protease-inhibiting peptide substrates. In a preferred embodiment, the protease-inhibiting peptide substrate is gelatin. The compositions may also contain a galactomannan. In a particularly preferred embodiment, the compositions contain gelatin, a galactomannan and a borate salt. The present invention also describes methods of use of these compositions to inhibit protease MMP-9, and methods of topical administration of the compositions to the eye, particularly for the treatment of dry eye.

The present application claims the benefit of U.S. Provisional Patent Application Ser. No. 60/988,623, filed on Nov. 16, 2007, the disclosure of which is specifically incorporated by reference herein.

### BACKGROUND OF THE INVENTION

Dry eye or xerophthalmia is a condition that causes pain and discomfort to many. For most individuals, blinking and replenishment of fluid throughout the day provide for a clean and conditioned eye surface. In dry eye, the surface of the eye becomes quite sensitive, and pain and irritation result. The etiology of dry eye is not known, although there are many theories as to the cause or causes of this condition. One theory posits a glandular defect, wherein the ocular glands that secrete fluids to replenish those lost to blinking, drainage and evaporation become deficient and secrete an inadequate quantity of fluid. Another possible cause of dry eye involves the nerves that populate the conjunctiva and cornea. These nerves either become desensitized, leading to less blinking and hence drying, or they become overly sensitized, leading directly to an increase in pain and irritation characteristic of dry eye symptomology. Chronic inflammation may be another causative or contributing factor to dry eye, whatever the origin of the inflammatory insult. Infection in the eyes can cause dry eye, and inflammation resulting from the infection can cause the tear ducts to become blocked. Autoimmune disorders, wherein the body mistakenly identifies its own tissues as being of foreign origin, may subject ocular tissues to immunological attack, and may also be a contributing factor to the etiology of dry eye. In one such autoimmune disorder, Sjogren's syndrome, dry eyes (and dry mouth) are among the hallmark symptoms caused by immune cells attacking the exocrine glands that produce tears and saliva. Sjogren's syndrome is estimated to afflict as many as four million people in the United States alone, making it the second most common autoimmune disease. Other possible causes of dry eye include hormonal or vitamin deficiencies or excesses. Dry eye may in fact be the result of a multiplicity of distinct conditions, any one or more of which may lead to the condition in any individual patient.

Whatever the causative factor(s), it is relief from the painful and debilitating symptoms that most dry eye patients are seeking. For that purpose, many approaches have been tried, ranging from surgical interventions to prescription pharmaceuticals to over-the-counter eyedrop products. Surgical options include removing normal drainage routes, either permanently or temporarily through occlusion of the lacrimal canal. For temporary occlusion, devices known as punctal plugs are utilized. Non-surgical devices developed to treat dry eye include humidity chambers used to augment eye moisture. Therapeutic agents, which may or may not be in form of eyedrops, seek to remedy the underlying physiological condition and thereby reduce the severity of dry eye, or eliminate it entirely. However, to date, only one therapeutic agent has been approved by the FDA for the treatment of dry eye. While each of these therapeutic or ameliorative approaches may provide benefits to certain patients, these approaches entail significant risk, expense, and/or inconvenience to the patient. A convenient, relatively low-cost and low risk treatment is available to dry eye sufferers in the form of artificial tear products. These topical agents are usually applied as eyedrops when needed to supplement or recondition the tear film. Thus, artificial tears are, in the most basic sense, simply another method of adding moisture to the eye. While they may provide symptomatic relief in some cases, they rarely alter any underlying ocular or corneal pathology.

One relatively recent line of research into the origin or etiology of dry eye examines the potential role of metalloproteinases in the cornea. Metalloproteinases are a group of proteolytic enzymes characterized by their need for a binding a metal ion, such as Zn²+ or Ca²+ in their active site in order to them to be catalytically active. Metalloproteinases, abbreviated as MMPs, are known to be involved in processes that involve tissue remodeling. Physiologically, therefore, MMPs play a role in tumor metastasis, embryonic development, and wound healing. There are about 20 known MMPs, all of which appear to be structurally related to each other, with about 40% amino acid homology. Historically, individual MMPs were given names based on what was thought to be their major substrate (for example, (i) collagenases, which degrade interstitial collagens (types I, II and III); (ii) type IV collagenases and gelatinases, which degrade basement membrane collagen type 4 and gelatins (denatured collagens); (iii) stromelysins, which degrade a broad range of substrates including proteoglycans, laminin, gelatins and fibronectin) or sometimes by the cellular source of the enzyme (for example, polymorphonuclear leukocyte gelatinase). Eventually it was accepted that most of these enzymes cleave multiple substrates, including the inactive polypeptide proforms(zymogen) of other family members, and that these enzymes can also degrade non-matrix proteins such as myelin basic protein and alpha-1-antitrypsin. Structurally, most MMPs have a catalytic domain, a carboxyterminal hemopexin-like domain (hemopexin domain), and a prodomain that is cleaved during enzyme activation,

An article published by H. Nagase et al in 1992 provided a numerical nomenclature and glossary of the MMPs known at that time (for example MMP-1, MMP-2, etc.) and later-discovered MMPs have followed that system. MMP-9 (gelatinase-B, collagenase type IV-B), as a physiological tissue remodeler, is active in degrading a broad range of extracellular matrix (ECM) and basement membrane components. MMP-9 appears to play a role in mediating inflammation, by converting inflammatory cytokine interleukin IL-1β into its active, secreted form, by catalyzing the postranslational activation of tumor necrosis factor (TNFα), by potentiating IL-8, processes chemokines, and by degrading serine protease inhibitors. In addition, MMP-9 may also play a role in autoimmunity, as it may promote the development of autoimmune neo-epitopes. The local activity of MMP-9 has been shown to be elevated in the tear fluid of patients with Sjogren's syndrome. Several studies have demonstrated a significant increase in the activity of gelatinases, including MMP-9, in the tear film of humans and other mammals with ulcerated keratitis as compared to the tear film of the healthy cornea. The role of gelatinases in the pathogenesis of ulcerative keratitis has also been investigated. A study with MMP-9 knockout mice has shown that a lack of MMP-9 confers some degree of resistance to corneal epithelial barrier disruption from experimentally-induced dry eye.

In their attempts to provide a therapeutic agent that acts to inhibit the activity of various MMPs in vivo, a large number of new chemical entities have been synthesized by many different research organizations. Several of these rationally designed MMP inhibitors passed a number of preclinical hurdles and showed potential as therapeutics for a number of the pathological conditions which are thought to involve MMPs. Unfortunately, several of these compounds, for example, marimastat (BB-2516), a broad spectrum MMP inhibitor, and trocade (Ro 32-3555), an MMP-1 selective inhibitor, have not performed as hoped in clinical trials. One reason attributed for their lack of success is significant side effects, such as musculo-skeletal toxicity, particularly with the broad spectrum inhibitors. A lack of disease-modifying efficacy is another issue, as in the case of trocade, where encouraging results in rabbit arthritis models were not duplicated in the trials conducted in humans. In fact, British Biotech's marimastat has been the subject of at least five failed Phase III trials, and both Bayer and Pfizer have terminated Phase III MMP inhibitor trials.

Recently, a novel gelatin binding site has been discovered as part of the hemopexin subunit of MMP-9.

WIPO Publication No. WO 95/2969 relates to compositions for tear replacement therapy containing cytokines or growth factors, particularly TGFβ8.

U.S. Pat. No. 6,444,791 (Quay) relates to a method for treating keratoconus using protease inhibitors, including alpha2-macroglobulin and alpha1-protease inhibitor.

U.S. Pat. No. 4,923,700 (Kaufman) relates to an artificial tear system including an aqueous suspension of mucin-type particles and lipid-type material. The mucin-type particles are formed from collagen, gelatin and/or serum.

U.S. Pat. No. 6,455,583 (Pflugfelder et al.) relates to the use of topical tetracycline to decrease inflammation associated with delayed tear clearance.

### SUMMARY OF THE INVENTION

It has now been surprisingly discovered that relatively small amounts of naturally occurring peptide protease inhibitors demonstrate significant inhibition of metalloproteinases when incorporated into ophthalmically acceptable vehicles, such as those used in artificial tear-type compositions. It has also been surprisingly discovered that the resulting compositions can act to increase the viability and decrease the desiccation of corneal epithelial cells. The present invention is directed to MMP-inhibiting topical ophthalmic compositions comprising a protease-inhibiting peptide substrate in an ophthalmically acceptable vehicle. The present invention is also directed to methods for treating dry eye comprising applying to an ocular surface a composition containing a protease-inhibiting peptide substrate in an ophthalmically acceptable vehicle.

A first group of embodiments of the present invention is directed to topical ophthalmic compositions comprising a protease-inhibiting peptide substrate and an ophthalmically acceptable vehicle. A preferred embodiment in this group of embodiments is a protease-inhibiting peptide substrate and a galactomannan in an ophthalmically acceptable vehicle. A further preferred embodiment is a topical ophthalmic composition comprising gelatin and a galactomannan. Another preferred embodiment is a composition of alpha-2-macroglobulin and galactomannan. Other embodiments of the present invention include compositions comprising galactomannan and ovomacroglobulin, galactomannan and collagen, and galactomannan and casein. A preferred galactomannan is HP-guar.

A second group of embodiments of the present invention is directed to a method of treating dry eye comprising applying to an ocular surface an effective amount of a MMP-9-inhibiting peptide substrate. In preferred embodiments here, the amount of peptide substrate is sufficient to inhibit MMP-9 by at least 50%.

Without wishing to be bound by theory, it is believed that the protease-inhibiting peptide substrates, acting to inhibit the activity of proteases such as MMP-9, thereby reduce the ability of proteases to act on the endogenous substrates normally present in ocular tissues subject to the dry eye disorder. In this way, they may act to reduce the directly damaging effects of MMP-9 or other ocular proteases. Some or all of the inhibitory effects of the protease-inhibiting peptide substrates on proteases such as MMP-9 may be indirect, that is, in the manner of an allosteric-type inhibition. The size or molecular weight of the protease-inhibiting peptide substrate may effect the potency of this inhibition. In addition, the protease-inhibiting peptide substrates may provide a direct or indirect antiinflammatory effect on the sensitized ocular surface tissues, as well as an anti-tissue remodeling effect. These actions are thought to be mediated by the interaction of the peptide substrates with MMP enzymes, particularly MMP-9. In addition, certain embodiments of the present invention may prolong these therapeutic actions by providing a sustained release of the protease inhibiting peptides. For example, in a preferred embodiment of the present invention, the protease-inhibiting peptide substrate is combined with HP-guar and borate to form a gel. This gel acts to enhance tear film stability and protect the ocular surface from dessication. Further, the gel can entrap the protease-inhibiting peptide substrate, and the substrates are thereby retained in the tear film, resulting in a prolonged duration of activity. The protease-inhibiting peptide substrates can also act as a scaffold for soluble mucin to form a gelatin-mucin gel matrix, thereby enhancing the stability of tear film.

### BRIEF DESCRIPTION OF THE DRAWINGS

FIG. 1 shows a dose response inhibition of MMP-9 by Gelatin A in Tricine buffer.

FIG. 2 shows that Gelatin A at 0.1% w/v in combination with demulcent polymers shows significant inhibition of MMP-9.

FIG. 3 shows a dose response inhibition of MMP-9 by Gelatin-A incorporated into Systane.

FIG. 4 shows a dose response inhibition of MMP-9 by Gelatin-A in Tears Naturale II.

FIG. 5 shows a dose response inhibition of bacterial collagenase by Gelatin-A.

FIG. 6 shows a dose response inhibition of bacterial collagenase by Gelatin-A in Systane.

FIG. 7 shows a dose response inhibition of bacterial collagenase by Gelatin-A in Tears Naturale II

FIG. 8 shows that Gelatin A in combination with demulcent polymers shows varying degrees of inhibition of bacterial collagenase.

FIG. 9 shows the increased dessication protection and viability of cells when treated with artificial tear products incorporating Gelatin A.

FIG. 10 shows a dose response inhibition of MMP-9 by α-2 macroglobulin.

FIG. 11 shows a dose response inhibition of MMP-9 by recombinant human gelatin 8.5 kD.

FIG. 12 shows a dose response inhibition of MMP-9 by recombinant human collagen.

### DETAILED DESCRIPTION OF THE INVENTION

As utilized herein, the following terms, unless otherwise indicated, shall be understood to have the following meanings:

The term “protease” encompasses enzymes that catalyze the cleavage of peptide bonds. Representative proteases include collagenase and matrix metalloproteinases.

The term “protease-inhibiting peptide substrate” encompasses substances that are primarily peptidic in nature, that is, composed of one or more amino-acid chains, and have the property of being a substrate for protease enzymes. Representative examples of protease-inhibiting peptide substrates include gelatin, alpha-two macroglobulin, ovomacroglobulin, casein and collagen.

The term “MMP” refers to a matrix metalloproteinase (enzyme).

The term “MMP-9” refers to the enzyme known as matrix metalloproteinase-9.

The term “galactomannan” refers to polysaccharides derived from natural gums or similar natural or synthetic gums containing mannose or galactose moieties, or both groups, as the main structural components.

The term “CMC” refers to carboxymethylcellulose and salts thereof.

The term “HPMC” refers to hydroxypropyl methylcellulose.

The term “HP-Guar” refers to hydroxypropyl guar. Hydroxypropyl guar with low molar substitution (e.g., less than 0.6) is preferred.

The term “ocular surface” refers to the externally accessible tissues of the eye, representative but non-limiting examples of which include the cornea, the conjunctiva, the fornix and the sclera.

The term “inhibiting amount” refers to a nontoxic but sufficient amount of the inhibiting substance to provide the desired activity.

The term “ophthalmically acceptable vehicle” means a composition having physical properties (e.g., pH and/or osmolality) that are physiologically compatible with ophthalmic tissues.

Surprisingly, it has been discovered that relatively small amounts of naturally occurring peptide protease inhibitors demonstrate significant inhibition of metalloproteinases when incorporated into artificial tear-type compositions. Even more surprisingly, the amount of protease-inhibiting peptide substrate can be quite low, for it has been found that concentrations of protease-inhibiting peptide substrate, one embodiment of which is gelatin, as low as 0.1% w/v can provide greater than 50% inhibition of MMP-9.

Exemplary protease-inhibiting peptide substrates include gelatin, alpha-2-macroglobulin, ovomacroglobulin, collagen and casein, and are further described below. However, it should be understood that other protease-inhibiting peptide substrates may be used and found to be within the scope of the present invention.

Gelatin is a protein produced by partial hydrolysis of collagen extracted from animal connective tissue. Two types of gelatin are commercially available: type A is derived from an acid-treated precursor, while type B is derived from an alkali-treated precursor. Both types of gelatin are substrates of various MMPs, and act as competitive inhibitors of MMPs.

Alpha-2 macroglobulin, a large protein produced by the liver and found in blood, is able to inactivate a number of proteinases, including metalloproteinases. The mechanism for this inactivation is reported to be a 35 amino acid region that acts as a ‘bait’ for the proteinase: when the proteinase binds and cleaves this region, it becomes bound to the alpha-2-macroglobulin. The resulting complex is then cleared from the blood by macrophages.

Casein is a phosphoprotein found in cheese and milk. Casein contains a relatively high number of proline residues, and as a result has little secondary or tertiary structure. While relatively hydrophobic, it is readily dispersible in dilute alkaline and salt solutions.

Ovomacroglobulin, also referred to as ovostatin, is a glycoprotein composed of four subunits joined in pairs by disulfide bonds. It has demonstrated broad-spectrum inhibitory activity against various types of proteases, including serine proteases, cysteine proteases, thiol proteases, and metalloproteases.

Collagen is the primary protein in animals, providing nearly 25% of the total protein content, and is the primary protein in connective tissue. It is a long fibrous protein, and forms tough bundles or fibers that together from the extracellular matrix that provides structure to tissues and cells. Collagen may also be found inside certain cells. Collagen is most commonly found in a triple helix form known as tropocollagen. It is the partial hydrolysis of tropocollagen that produces gelatin.

The source of the protease-inhibiting peptide substrates utilized in the present invention is typically of animal origin. For example, gelatin derived from bovine or porcine skin or bone is the predominant form used in pharmaceutical products today. Extensive processing is undertaken in order to provide as homogenous and pure a product as possible, given the intended use (oral, parenteral, device). Collagen and/or gelatin that is free of transmissible spongiform encephaly (TSR) and Bovine spongiform encephalopathy (BSE) is commercially available from a number of suppliers, including, for example, Gelita (Sergeant Bluff, Iowa) and Rousselot (a Sobel Company, Dubuque, Iowa). Use of materials produced via synthetic and/or recombinant technology is another option. For example, Fibrogen (San Francisco, Calif.) produces fully synthetic gelatins and collagens using a recombinant yeast system. These synthetic materials may have some advantages in terms of consistency (lot-to-lot uniformity, defined molecular weight and physico-chemical properties), customizability (predetermined characteristics, designer molecules) and biocompatibility and safety (reduced risk of inducing an immune response, elimination of contaminants).

The compositions and methods of the present invention include protease-inhibiting peptide substrates in an amount sufficient to inhibit metalloproteinases. The preferred metalloproteinase is MMP-9. The amount of protease-inhibiting peptide substrate may vary depending on the specific substrate, but in general the amount is from about 0.010% to 10% weight/volume (w/v), more preferably, from about 0.05% to 1.0% (w/v), more preferably still from about 0.05% to 0.25% (w/v). The percent degree of inhibition of MMP is preferably more than about 50%, more preferably, more than about 60%, more preferably still, more than about 70%.

In one embodiment of the present invention, the protease-inhibiting peptide substrate is combined with an existing dry eye formulation such as SYSTANE® Lubricant Eye Drops (Alcon Laboratories, Inc.), which contain a lubricating polymer system. The polymerizing protection of SYSTANE® is achieved through the interaction of the demulcents (polyethylene glycol 400 and propylene glycol), HP-Guar and the patient's natural tears. When HP-Guar combines with natural tears, a chemical reaction occurs. HP-Guar binds to the hydrophobic (water repellant) surface, forming a network with a gel-like consistency. HP-Guar also helps keep the demulcent system on the surface of the eye longer.

One embodiment of the present invention is a composition combining a galactomannan, borate and gelatin. The types of galactomannans that may be used in the present invention are typically derived from guar gum, locust bean gum and tara gum. Additionally, the galactomannans may also be obtained by classical synthetic routes or may be obtained by chemical modification of naturally occurring galactomannans.

As used herein, the term “galactomannan” refers to polysaccharides derived from the above natural gums or similar natural or synthetic gums containing mannose or galactose moieties, or both groups, as the main structural components. Preferred galactomannans of the present invention are made up of linear chains of (1-4)-.beta.-D-mannopyranosyl units with .alpha.-D-galactopyranosyl units attached by (1-6) linkages. With the preferred galactomannans, the ratio of D-galactose to D-mannose varies, but generally will be from about 1:2 to 1:4. Galactomannans having a D-galactose:D-mannose ratio of about 1:2 are most preferred. Additionally, other chemically modified variations of the polysaccharides are also included in the “galactomannan” definition. For example, hydroxyethyl, hydroxypropyl and carboxymethylhydroxypropyl substitutions may be made to the galactomannans of the present invention. Non-ionic substitutions to the galactomannans, such as those containing alkoxy and alkyl (C1-C6) groups are particularly preferred when a soft gel is desired (e.g., hydroxylpropyl substitutions). Substitutions in the non-cis hydroxyl positions are most preferred. An example of a composition formed by non-ionic substitution of a galactomannan is hydroxypropyl guar, with a molar substitution of about 0.4. Anionic substitutions may also be made to the galactomannans. Anionic substitution is particularly preferred when strongly responsive gels are desired.

Borate compounds may be used with certain embodiments of the present invention. The borate compounds which may be used in compositions of the present invention include boric acid and other pharmaceutically acceptable salts such as sodium borate (borax) and potassium borate. As used herein, the term “borate” refers to all pharmaceutically suitable forms of borates. Borates are common excipients in ophthalmic formulations due to good buffering capacity at physiological pH and well known safety and compatibility with a wide range of drugs and preservatives. Borates also have inherent bacteriostatic and fungistatic properties, and therefore aid in the preservation of the compositions.

A preferred embodiment of the present invention is a composition comprising gelatin in the amount of 0.01% to 5% (w/v), one or more galactomannan(s) in the amount of from about 0.1 to 5% (w/v) and borate in the amount of from about 0.05 to 5% (w/v). Preferably, the compositions will contain 0.01% to 1.0% gelatin (w/v), 0.2 to 2.0% (w/v) of galactomannan and 0.1 to 2.0% (w/v) of a borate compound. Most preferably, the compositions will contain 0.05 % to 0.5% gelatin (w/v), 0.3 to 0.8% (w/v) of galactomannan and 0.25 to 1.0% (w/v) of a borate compound. The particular amounts will vary, depending on the particular gelling properties desired. In general, the gelatin, borate or galactomannan concentration may be manipulated in order to arrive at the appropriate viscosity of the composition upon gel activation (i.e., after administration). Manipulating either the gelatin, borate or galactomannan concentration may provide stronger or weaker gelation at a given pH. If a strongly gelling composition is desired, then the gelatin, borate or galactomannan concentration may be increased. If a weaker gelling composition is desired, such as a partially gelling composition, then the gelatin borate or galactomannan concentration may be reduced. Other factors may influence the gelling features of the compositions of the present invention, such as the nature and concentration of additional ingredients in the compositions, such as salts, preservatives, chelating agents and so on. Generally, preferred non-gelled compositions of the present invention, i.e., compositions not gel-activated by the eye, will have a viscosity of from about 5 to 1000 cps. Generally, preferred gelled compositions of the present invention, i.e., compositions gel-activated by the eye, will have a viscosity of from about 50 to 50,000 cps.

One of the earliest and most successful artificial tear solutions is described in U.S. Pat. No. 4,039,662 (Hecht, et al.). This solution has been marketed for many years as TEARS NATURALE™ Lubricant Eye Drops (Alcon Laboratories, Inc., Fort Worth, Tex.). The solution, described and claimed in the Hecht, et al. '662 patent, and the corresponding commercial product are based on the use of a unique combination of hydroxypropyl methylcellulose, Dextran 70 and benzalkonium chloride. In a later version of this product, which is currently marketed under the name TEARS NATURALE™ II Polyquad® Lubricant Eye Drops (Alcon Laboratories, Inc.), the benzalkonium chloride was replaced by polyquaternium-1, which is a polymeric antimicrobial agent/preservative.

An example of an organic buffer that may be utilized in the present invention is Tricine, or N-[tris(hydroxymethyl)methyl]glycine. Organic buffer have both basic and acidic groups, and as a result are zwitterionic; under physiologic pH conditions, these buffers carry both a positive and a negative charge.

In the case of contact lens and ophthalmic solutions, various agents are added to enhance compatibility with the eye. To avoid stinging or irritation it is important that the solution possess a tonicity and pH within the physiological range, e.g., 200-350 mOsmole for tonicity and 6.5-8.5 for pH. To this end, various buffering and osmotic agents are often added. The simplest osmotic agent is sodium chloride since this is a major solute in human tears. In addition propylene glycol, lactulose, trehalose, sorbitol, mannitol or other osmotic agents may also be added to replace some or all of the sodium chloride. Also, various buffer systems such as citrate, phosphate (appropriate mixtures of Na.sub.2 HPO.sub.4, NaH.sub.2 PO.sub.4, and KH.sub.2 PO.sub.4), borate (boric acid, sodium borate, potassium tetraborate, potassium metaborate and mixtures), bicarbonate, and tromethamine and other appropriate nitrogen-containing buffers (such as ACES, BES, BICINE, BIS-Tris, BIS-Tris Propane, HEPES, HEPPS, imidazole, MES, MOPS, PIPES, TAPS, TES, Tricine) can be used to ensure a physiologic pH between about pH 6.5 and 8.5.

The protease-inhibiting peptide substrate compositions of the invention may be combined with one or more additional therapeutic agents from other therapeutic classes believed to have beneficial effects in treating dry eye, such as, for example, antibiotics, immunosuppressants, and antiinflammatory agents.

Antiinflammatory agents that may be included in the compositions of the invention include steroidal or non-steroidal drugs (NSAIDs). Exemplary NSAIDs include, but are not limited to, ketorolac tromethamine (Acular®), indomethacin, flurbiprofen sodium, nepafenac, bromfenac, suprofen and diclofenac (Voltaren®). Exemplary corticosteroids include, but are not limited to, rimexoline, hydrocortisone, fludrocortisone, fluoromethalone, loteprednol, triamcinolone, dexamethasone, prednisolone, cortisone, aldosterone, mydrysone and betamethasone. Exemplary sex steroids include those based upon androgens, estrogens, and/or progestins.

Exemplary antibiotics include, but are not limited to, tetracycline, doxycycline, and chemically-modified tetracyclines, beta-lactam antibiotics, such as cefoxitin, n-formamidoylthienamycin and other thienamycin derivatives, chloramphenicol, neomycin, carbenicillin, colistin, penicillin G, polymyxin B, vancomycin, cefazolin, cephaloridine, chibrorifamycin, gramicidin, bacitracin, sulfonamides enoxacin, ofloxacin, cinoxacin, sparfloxacin, thiamphenicol, nalidixic acid, tosufloxacin tosilate, norfloxacin, pipemidic acid trihydrate, piromidic acid, fleroxacin, chlortetracycline, ciprofloxacin, erythromycin, gentamycin, norfloxacin, sulfacetamide, sulfixoxazole, tobramycin, moxifloxacin and levofloxacin.

Exemplary immunosuppressives include, for example, cyclosporins such as cyclosporin A and ascomycins such as FK-506, rapamycin and tacrolimus.

Other ingredients may be added to the compositions of the present invention. Such ingredients generally include tonicity adjusting agents, chelating agents, active pharmaceutical agents, solubilizer, preservatives, pH adjusting agents and carriers. Other polymer or monomeric agents such as polyethylene glycol and glycerol may also be added for special processing. Tonicity agents useful in the compositions of the present invention can include salts such as sodium chloride, potassium chloride and calcium chloride; non-ionic tonicity agents may include propylene glycol and glycerol; chelating agents may include propylene glycol and glycerol; chelating agents may include EDTA and its salts; solublizing agents may include Cremophor EL® and tween 80; other carriers may include Amberlite® IRP-60; pH adjusting agents may include hydrochloric acid, Tris, triethanolamine and sodium hydroxide; and suitable preservatives may include benzalkonium chloride, polyquaternium-1 and polyexamethylene biguanide. The above listing of examples is given for illustrative purposes and is not intended to be exhaustive. Examples of other agents useful for the foregoing purposes are well known in ophthalmic formulations and are contemplated by the present invention.

The following examples further illustrate various embodiments of the invention. These examples are provided to aid in the understanding of the invention and are not to be construed as limitations thereof.

### EXAMPLE 1

In this and the following examples, unless otherwise indicated, MMP activity was assessed using fluorogenic substrates susceptible to MMP-1, -2, and -9, including DNP-Pro-Leu-Gly-Met-Trp-Ser-Arg-OH and DNP-Pro-Cha-Gly-Cys(Me)-His-Ala-Lys(N-Me-Abz)-NH₂. These fluorogenic substrate assays are well known in the art; for example, see Bickett et al. Analytical Biochemistry 212, 58-64 (1993) and Netzel-Arnett et al., Analytical Biochemistry 195, 86-92 (1991), both of which are hereby incorporated into this disclosure by reference. Before conducting the assay the pro-MMP-9 was activated by p-aminophenylmercuric acetate and no activation of bacterial collagenase was required. For assay a stock solution of the substrate at 0.1 mM concentration in DMSO was prepared and all of the enzyme activity assays with or without inhibitors were performed in 50 mM tricine buffer, pH 7.5, containing 0.2M NaCl, 10 mM CaCl2, 50 mM ZnSO4, and 0.05% Brij-35 at room temperature. (Brij-35 is a commercially available polyoxyethylene lauryl ether surfactant). The total sample volume was 200 μl and was conducted in a 96-well microplate. The fluorescence changes were recorded every minute for 10 minutes with a microplate fluorescence reader (Model FL x8001, Bio-Tek Instrument) setting at a proper excitation/emission wavelength (i.e. λex=280 nm; λem=360 nm and λex=280 nm; λem=360 nm) for the specific substrate that was being used. The activity of enzyme was expressed as the fluorescence change per minute which was the slope of the linear line regarding the fluorescence versus time recorded for the enzyme reaction within the 10 minutes. The % inhibition was calculated by subtracting the rate of the inhibitor sample from the rate of sample without inhibitor and then dividing by the rate of sample without inhibitor multiplying 100%.

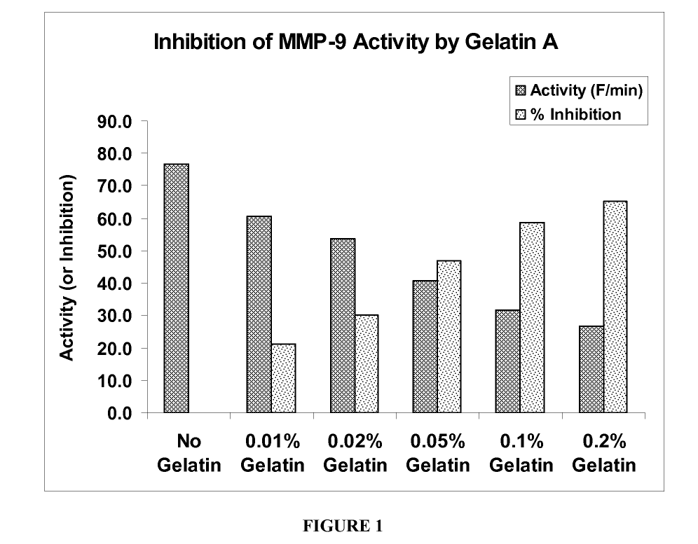

This study was undertaken to investigate the potential of Gelatin A to inhibit MMP-9 activity. In this particular study, the concentration of MMP-9 was 360 μUnits/assay in Tricine buffer, the gelatin used was Gelatin A (Sigma Catalog #1890-50G, Lot #014K0077, acid extract from porcine skin), and the substrate used was MMP-2/MMP-9 fluorogenic substrate I (Calbiochem Catalog #44215, lot #B47246; peptide structure=DNP-Pro-Leu-Gly-Met-Trp-Ser-Arg-OH.) 20 μM/assay. Gelatin A in Tricine buffer showed a dose response inhibition of MMP-9 activity. Starting from 0.01% up to 0.2% (w/v) a proportional increase of inhibition was observed. After 0.2%, the inhibition started to level off. The results of the study are described graphically in FIG. 1.

### EXAMPLE 2

This study was undertaken to investigate the potential of Gelatin A to inhibit MMP-9 activity when used with various demulcent polymers. For this purpose 0.1% w/v Gelatin A that provided approximately 59% inhibition from Example 1 was chosen. MMP-9=Calbiochem Cat# 444231;Lot# B56458; human neutrophil. Activity used was 200 μunits/assay. Gelatin=Gelatin A. Sigma Cat# 1890-50G, Lot# 014K0077 (an acid extract from porcine skin). Assay Buffer=50 mM Tricine, pH 7.5, containing 0.2M NaCl, 10 mM CaCl2. Substrate=MMP-1/MMP-9 fluorogenic substrate. Calbiochem cat# 44221, lot# B54710. MWt, 1077.2; 1 μM in assay. Peptide structure=DNP-Pro-Cha-Gly-Cys(Me)-His-Ala-Lys(N-Me-Abz)-NH2. Ex, 365 nm; Em, 450 nm.

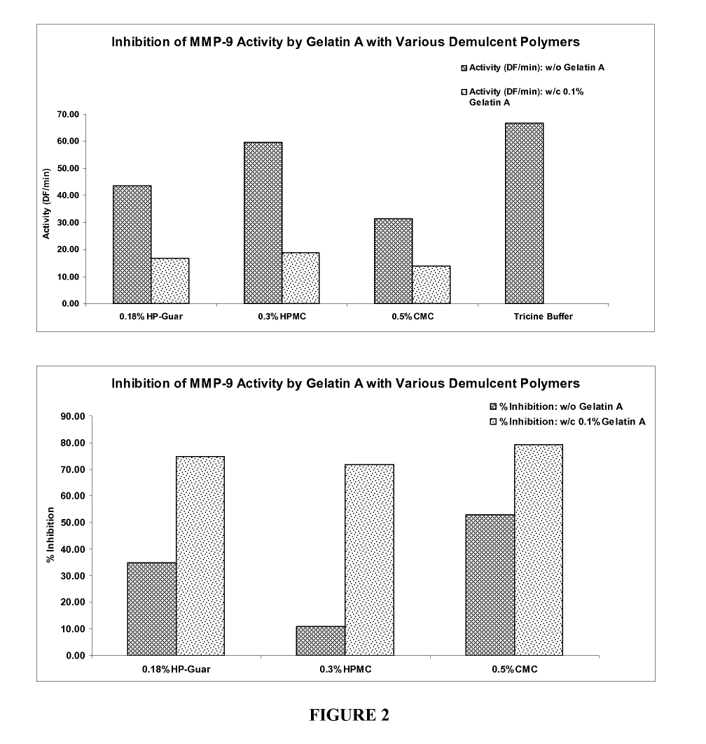

The results of this study show that Gelatin A at 0.1% w/v, in combination with 0.18% w/v HP-guar, 0.3% w/v HPMC and 0.5% w/v CMC, was able to provide 74.8%, 71.8% and 79.1 % inhibition of MMP-9 respectively, as shown in FIG. 2.

### EXAMPLE 3

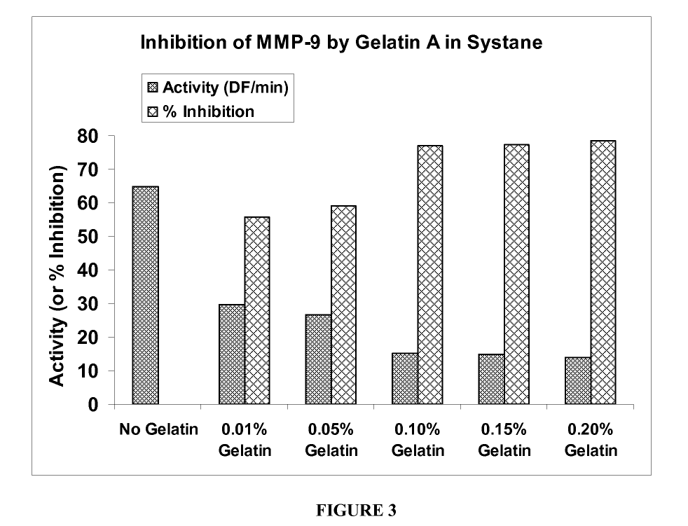

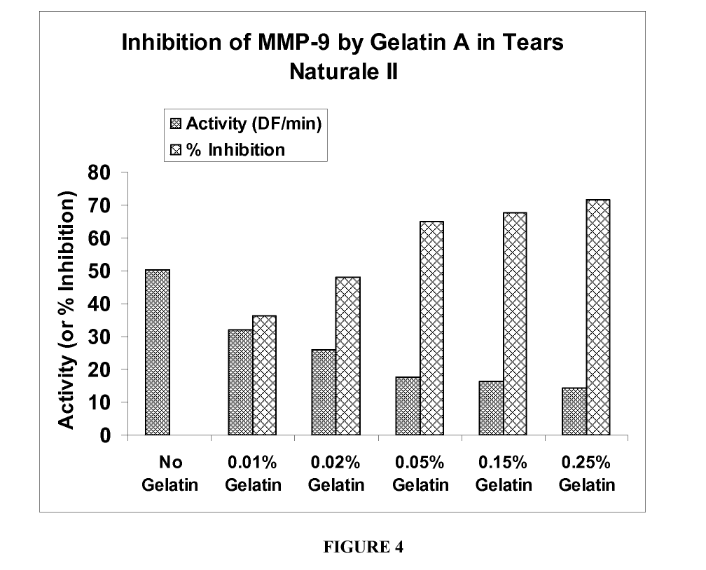

The next series of studies investigated the ability of Gelatin-A to inhibit MMP-9 activity when incorporated into two representative artificial tear solutions. For this purpose the artificial tear solutions known as Systane and Tears Naturale II were chosen. For this series of studies various concentrations of gelatin-A ranging from 0.01% to 0.20% (w/v) were incorporated into both of the marketed Systane and Tears Naturale II solutions. The assay was conducted using the same enzyme and substrate, and following the same procedure as described in Example 2. The results of this study demonstrate that Gelatin-A showed a dose response inhibition of MMP-9 activity when incorporated into both Systane and Tears Naturale II solutions, contributing more than 50% inhibition from 0.01% w/v and up in Systane, and from 0.05% w/v and up in Tears Naturale II. The results of these studies are described graphically in FIGS. 3 and 4.

### EXAMPLE 4

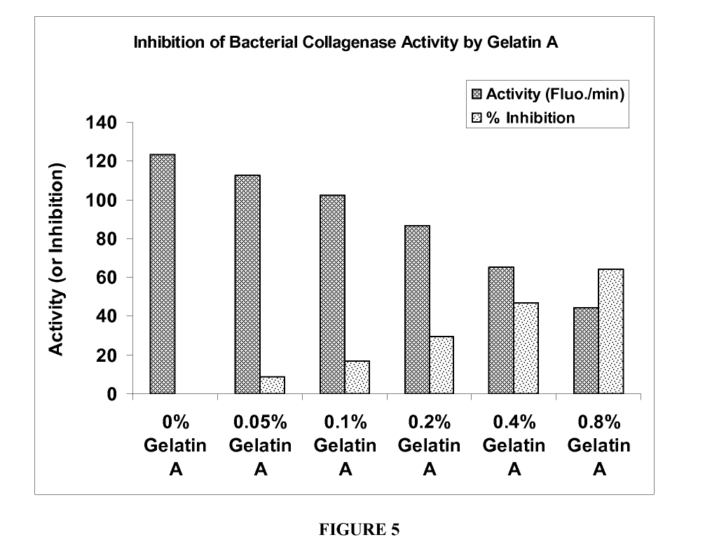

This study was undertaken to investigate the inhibition reactivity of Gelatin A on bacterial collagenase. Bacterial collagenases are exotoxins that assist in destroying extracellular structures in bacteria pathogenesis. For the study various concentrations of gelatin A ranging from 0.05% to 0.8% (w/v) were prepared in 50 mM tricine buffer pH 7.5, containing 0.2 M NaCl, 10 mM CaCl₂, ZnSO₄, and Brij-35. The activity of bacterial collagenase was assayed by recording the fluorescence change for 10 min with a spectrofluorometer at 25° C. The activity was expressed as the fluorescence change per min. The concentrations of bacterial collagenase & substrate I were 20 Units/assay and 20 μM/assay respectively. Collagenase used was Clostridopeptidase (Sigma Catalog #C-7657; lot #107H8632). Gelatin used was Gelatin A (Sigma Cat #1890-50G, Lot #014K0077. Acid extract from porcine skin). Substrate used was MMP-2AMMP-9 fluorogenic substrate I (Calbiochem cat #44215, lot #B47246; Peptide structure =DNP-Pro-Leu-Gly-Met-Trp-Ser-Arg-OH). The results indicate that it more than 0.4% w/v Gelatin A was required to exert an over 50% inhibition on the bacterial enzyme, whereas in the range of 0.05% to 0.1% (w/v) gelatin A could easily provide greater than 50% inhibition on MMP-9. Thus, inhibition of Gelatin A on bacterial collagenase seemed not as effective as that on MMP-9. The results of this study are described graphically in FIG. 5.

### EXAMPLE 5

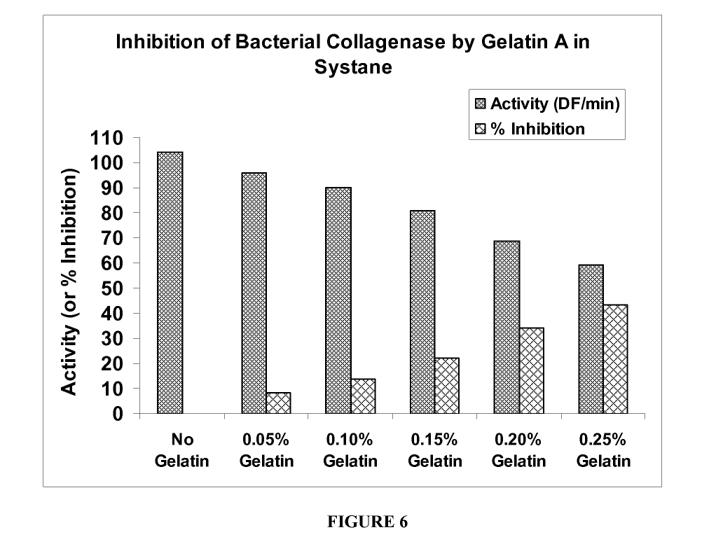

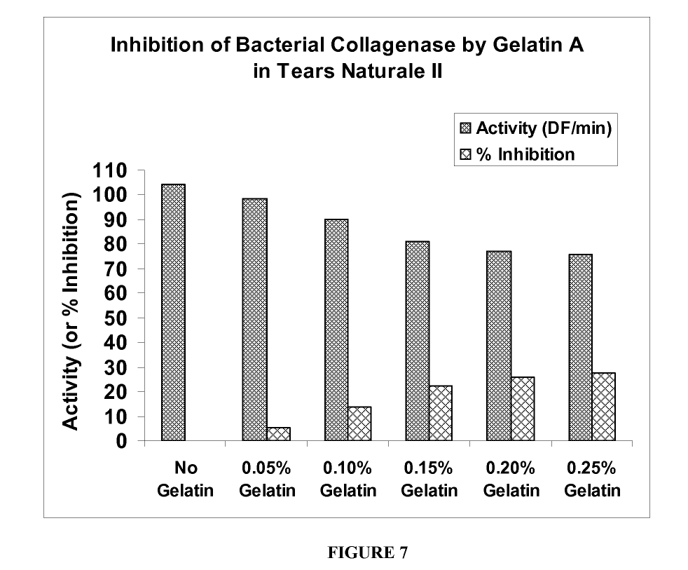

This study tested the ability of Gelatin A to inhibit bacterial collagenase when incorporated into artificial tear products. For this series of studies various concentrations of gelatin-A ranging from 0.05% to 0.25% (w/v) were incorporated into both marketed Systane and Tears Naturale II. The assay was carried out by the same procedure and using the same substrate as described in Example 4. The results show that Gelatin A, when incorporated into artificial tear products Systane and Tears Naturale II, could also provide a dose response inhibition on bacterial collagenase. However, it was less effective and required more than 0.25% w/v of gelatin to reach 50% inhibition in both artificial tears, as shown graphically in FIGS. 6 and 7.

### EXAMPLE 6

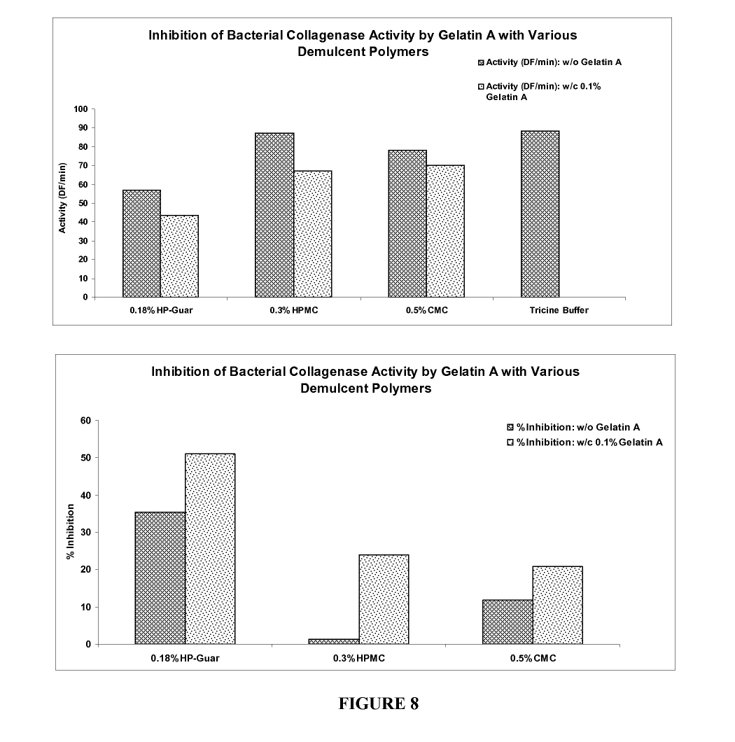

This study was undertaken to investigate the potential of Gelatin A to inhibit bacterial collagenase activity when used with various demulcent polymers. For the purpose Gelatin A at 0.1 % w/v was combined with HP-Guar, CMC and HPMC. The study was conducted by the same procedure and using the same substrate as described in Example 4. The results showed at with 0.18% HP-guar, 51% inhibition was attained, while with 0.3% HPMC and 0.5% CMC, only 24% and 21% inhibition were attained respectively. These results are shown graphically in FIG. 8.

### EXAMPLE 7

These studies looked at the ability of Gelatin A to provide dessication protection and to enhance the viability of Human corneal epithelial cells. In these studies, CEPI 17 Human corneal epithelial cells were assayed using an Alamar Blue method described here. A human corneal epithelial cell line (CEPI 17, Alcon Laboratories Inc.) was grown to confluency in the 96-well microplate. Medium was removed from the test wells and 100 μl of each test solution was added. Control wells with the medium were left alone. The plate was placed back in the incubator for 60 minutes. After incubation, all the wells were aspirated and rinsed once with 200 μl per well with HyQ buffer (A modified Dulbecco's phosphate buffered solution, Hyclone cat# SH30028.02). A 1/10 dilution of Alamar Blue (Biosource, DAL 1100) in HyQ was made and 100 μl was added to each well to incubate at 37° C. After 4 hours incubation, the plate was read by a fluorescence microplate reader (Model FLx800, Bio-Tek Instrument) with a setting of excitation at 560 nm and emission at 590 nm. Calculation of the % cell viability was carried out by dividing the average fluorescence of the sample by the average fluorescence of the control multiplied by 100%.

To assess the dessication protection, a similar procedure is used with a 15 minute pre-incubation and a 30 minute desiccation period. After pre-incubation, all the test wells except the controls were aspirated. The controls were covered with parafilm. The plate was placed in a downward air-flow hood for 30 minutes to expose the cells for desiccation. After desiccation, all wells were washed one time with 200 μl of HyQ. Cells were analyzed for viability by the Alamar Blue assay as described in the cell viability assay procedure. Calculation of the desiccation protection was carried out by dividing the average fluorescence of the sample by the average fluorescence of the control multiplied by 100%.

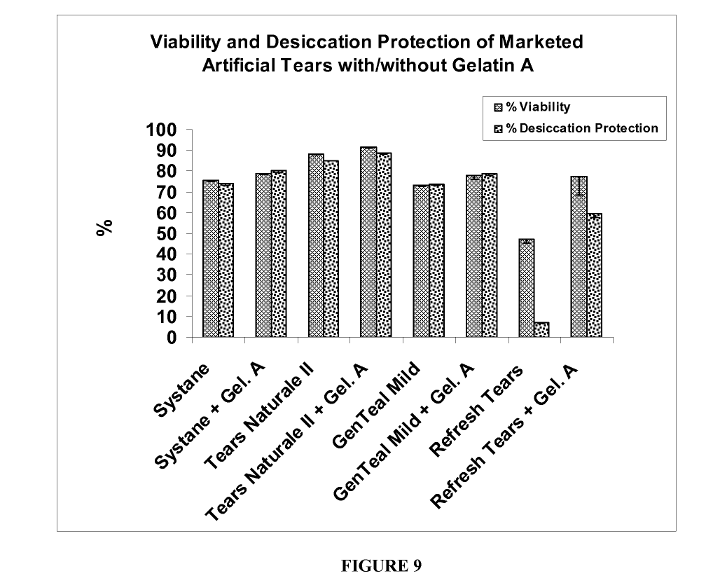

The results of this study demonstrate that incorporation of Gelatin A into various artificial tear products, including Systane, Tears Naturale II and GenTeal Mild, seems to provide better desiccation protection of the cells and to enhance the cell viability. The results of this study are shown graphically in FIG. 9.

### EXAMPLE 8

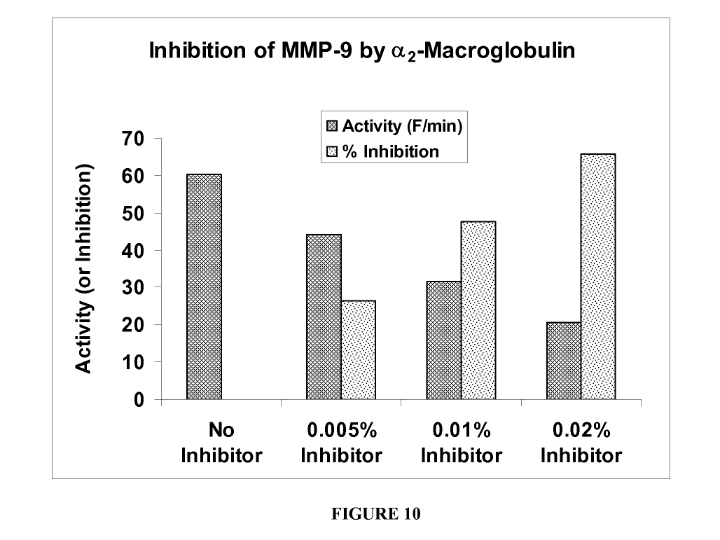

This study was undertaken to examine the ability of alpha-2-macroglobulin to inhibit MMP-9 activity. The activity of MMP-9 was assayed by recording the fluorescence change for 10 min with a spectrofluorometer at 25° C. The activity was expressed as the fluorescence change per min. 360 μUnits/assay of MMP-9 was used (Calbiochem Cat #444231, Lot #B56458; human neutrophil). α2-Macroglobulin (Sigma Cat #M-6159, Lot #118H7606; from human placenta). Substrate used was MMP-2/MMP-9 fluorogenic substrate I 10 μM/assay (Calbiochem cat #44215, lot #B47246; Peptide structure=DNP-Pro-Leu-Gly-Met-Trp-Ser-Arg-OH.) The results shows that alpha-2-macroglobulin inhibits MMP-9 activity in a dose response fashion. The results are described graphically in FIG. 10.

### EXAMPLE 9

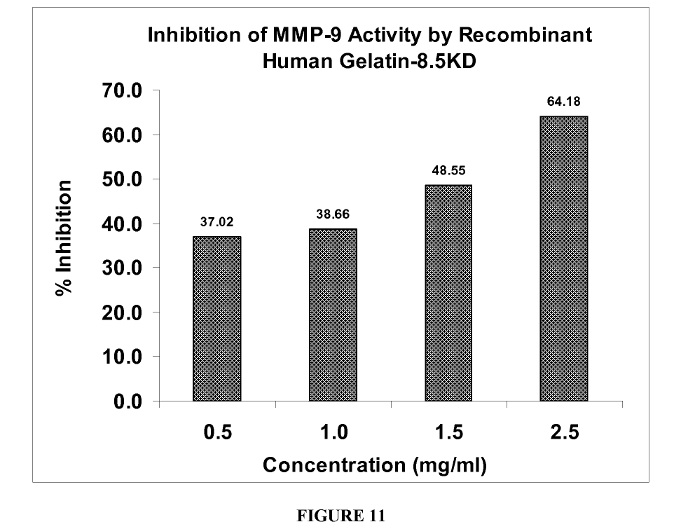

This study was undertaken to examine the effect of recombinant gelatin of known size to inhibit MMP-9 activity. The activity of MMP-9 was assayed by recording the fluorescence change for 10 min with a spectrofluorometer at 25° C. The activity was expressed as the fluorescence change per min. The concentration of MMP-9 (Calbiochem Catalog # 444231, lot #B56458, human neutrophil) was 200 μUnits/assay in Tricine buffer (50 mM Tricine, pH 7.5, containing 0.2M NaCl, 10 mM CaCl₂). The gelatin used was recombinant human gelatin 8.5 kD (FibroGen, Lot #04AE001R-01). Substrate used was MMP-1/MMP-9 fluorogenic substrate (Calbiochem Catalog #44221, lot #B54710; peptide structure=DNP-Pro-Cha-Gly-Cys(Me)-His-Ala-Lys(N-Me-Abz)-NH₂; Ex 365 nm; Em 450 nm). 1.0 μM/assay was used. Between 0.15% to 0.25% (w/v) Recombinant human gelatin 8.5 kD was required in this assay to achieve more than 50% inhibition. The results of the study are described graphically in FIG. 11.

### EXAMPLE 10

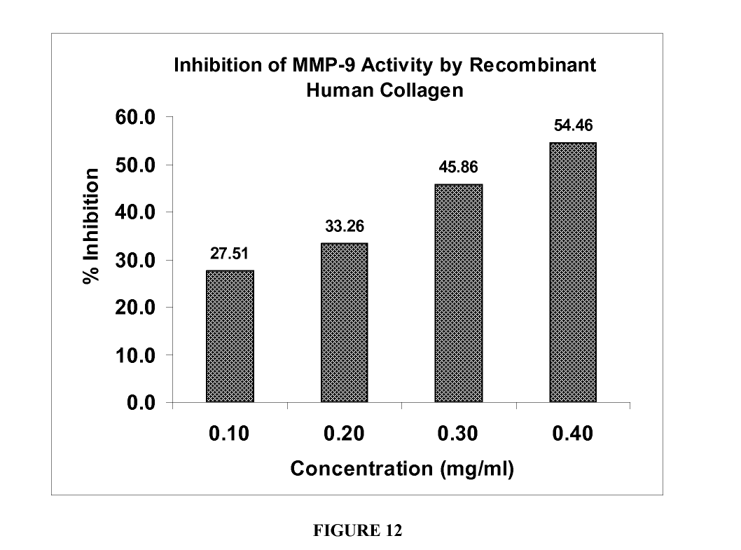

This study was undertaken to examine the effect of recombinant Human Collagen Type I to inhibit MMP-9 activity. The activity of MMP-9 was assayed by recording the fluorescence change for 10 min with a spectrofluorometer at 25° C. The activity was expressed as the fluorescence change per min. The concentration of MMP-9 (Calbiochem Catalog # 444231, lot #B56458, human neutrophil) was 200 μUnits/assay in Tricine buffer (50 mM Tricine, pH 7.5, containing 0.2M NaCl, 10 mM CaCl₂). The collagen used was recombinant Human Collagen Type I (FibroGen, Lot #04AE001R-01). Substrate used was MMP-1/MMP-9 fluorogenic substrate (Calbiochem Catalog #44221, lot #B54710; peptide structure=DNP-Pro-Cha-Gly-Cys(Me)-His-Ala-Lys(N-Me-Abz)-NH₂; Ex 365 nm; Em 450 nm). 1.0 μM/assay was used. Between 0.03% to 0.04% (w/v) recombinant Human Collagen Type I was required in this assay to achieve more than 50% inhibition. The results of the study are described graphically in FIG. 12.

### EXAMPLE 11

The following is an example of two artificial tear solutions of the present invention.

| | Amount % (w/v) | |
|---|---|---|
| Compound | Systane | Tears Naturale II |
| Gelatin | 0.1 | 0.1 |
| Boric Acid | 1.0 | n/p |
| Sodium Borate | n/p | 0.35 |
| HPMC | n/p | 0.3 |
| Hydroxypropyl Guar | 0.18 | n/p |
| Propylene Glycol | 0.3 | n/p |
| PEG-400 | 0.4 | n/p |
| Dextran 70 | n/p | 0.1 |
| Sodium Chloride | 0.1 | 0.6 |
| Potassium Chloride | 0.12 | 0.12 |
| Calcium Chloride (dehydrate) | 0.0053 | n/p |
| Magnesium Chloride (hexahydrate) | 0.0064 | n/p |
| Polyquaternium-1 | 0.001 | 0.001 |
| Sodium Hydroxide/Hydrochloric | to pH 7.0 | to pH 7.4 |
| Acid | | |
| Purified Water | qs to 100% | qs to 100% |

We claim

1. A topical ophthalmic composition comprising a protease-inhibiting peptide substrate in an ophthalmically acceptable vehicle.

2. A composition according to claim 1, further comprising a galactomannan.

3. A composition according to claims 1 or 2 wherein the protease-inhibiting peptide substrate is capable of inhibiting the protease MMP-9.

4. A composition according to claims 1, 2 or 3 wherein the protease-inhibiting peptide substrate is selected from the group consisting of gelatin, alpha-2-macroglobulin, ovomacroglobulin, collagen and casein.

5. A composition according to claim 2 wherein the galactomannan comprises HP-guar.

6. A method for treating dry eye which comprises applying to an ocular surface a composition containing an effective amount of a protease-inhibiting peptide substrate.

7. A method for treating dry eye which comprises applying to an ocular surface a composition containing an amount of a protease-inhibiting peptide substrate effective to inhibit MMP-9

8. A method according to claims 6 or 7 wherein the protease-inhibiting peptide substrate is selected from the group consisting of gelatin, alpha-2-macroglobulin, ovomacroglobulin, collagen and casein.

9. A method for treating dry eye which comprises applying to an ocular surface a composition comprising an effective amount of a protease-inhibiting peptide substrate and a galactomannan.

10. A method for treating dry eye which comprises applying to an ocular surface a MMP-9-inhibiting amount of a composition according to claim 1.

11. A composition according to claim 1, wherein the protease-inhibiting peptide substrate is present in an amount greater than about 0.05% and less than about 0.25%

12. A composition according to claim 11, wherein the protease-inhibiting peptide substrate is gelatin present in an amount greater than about 0.05% w/v.

13. A composition according to claim 11, wherein the protease-inhibiting peptide substrate is collagen present in an amount greater than about 0.03% w/v.

14. A composition according to claim 11, wherein the substrate is alpha-2-macroglobulin present in an amount greater than about 0.0 15% w/v.

15. A composition according to any of claims 1, 2, 3, 4, 5 or 11, further comprising a therapeutic agent selected from the group consisting of antibiotics, antiinflammatory agents and immunosuppressants.
